(19) United States
(12) Patent Application Publication
(10) Pub. No.: US 2009/0082756 A1
(43) Pub. Date: Mar. 26, 2009

(54) Transradial coronary catheter

(76) Inventor: Vasundhara Vidyarthi, Great Neck, NY (US)

Correspondence Address: RYAN KROMHOLZ & MANION, S.C., POST OFFICE BOX 26618, MILWAUKEE, WI, 53226, US

(21) Appl. No.: 11/903,959

(22) Filed: Sep. 25, 2007

(51) Int. Cl. A61M 25/098; A61M 25/00

(52) U.S. Cl. 604/527; 604/532

(57) ABSTRACT

Provided is a catheter suitable for catheterization of a right coronary artery using a right transradial approach. The catheter presents improved directionality, thereby requiring minimized external torque to be applied during insertion and diagnostic procedures. A catheter according to the disclosed design includes a tip near its distal end, a primary curve, a secondary curve, a tertiary curve and a proximal end accessible external a catheterized body. When properly inserted, the secondary curve may rest near the junction of the bracheocephalic trunk and the right subclavian artery, and the tertiary curve may rest in the superior curve of the right subclavian artery. In addition to improved directionality, the secondary and/or tertiary curve offers additional resistance against cephalic, or upward, back-up force by providing caudal, or downward, torque to more distal portions of the catheter body.

### BACKGROUND OF THE INVENTION

The present invention relates generally to catheters and more specifically to a catheter for use in a right transradial approach to the ostium of a right coronary artery. P Catheterization of a coronary artery is necessary to perform a variety of procedures, such as coronary angiography and angioplasty. Catheters are usually inserted into a body at a point remote from the heart, such as through an arterial access point in an arm or a leg. Access through a leg is often provided through a femoral artery. Through an arm, there are a few access options, including the brachial artery and the radial artery. P Access to the heart through a radial artery, that is, access using a transradial approach, is often favored due to a lower risk of vascular complications and ease of patient ambulation. For example, with a femoral approach, a patient is required to remain immobilized for a period of about four to six hours to ensure that bleeding from the access point has ceased. A brachial approach, though it lessens the patient's period of rest to one to two hours, prevents blood flow to the lower arm, wrist and hand during the procedure, and increases vascular complications due to the relative depth of the artery beneath the skin. P A popular catheter that has been, and still is, employed for cannulating the right coronary artery is known as the Judkins right catheter, named after Dr. Melvin P. Judkins. The curvature, that is, inverse first and second curves, of the Judkins right catheter is well suited to engage the right coronary artery ostium when the catheter is inserted femorally. Despite its design being particularly suited for a transfemoral approach, the Judkins catheter has also been used in a transradial approach. However, when the catheter is inserted through the right radial artery into the arch of the aorta, an external torque is required to engage the tip of the catheter with the right coronary artery ostium. Most often, this externally applied torque must be maintained with both hands of the doctor performing the procedure as the tendency is for the catheter to disengage from the ostium towards the left coronary cusp of the aortic valve. This makes such a procedure especially difficult if only a single doctor is performing the right transradial catheterization. P Another catheter used in a transradial approach is disclosed in U.S. Pat. No. 6,355,026, to Mick, which is incorporated herein by reference in its entirety. Mick recognizes the disadvantages of prior catheters mentioned above and provides a catheter specifically for use in a right transradial approach to the right coronary artery ostium. P While the Mick design accounts for some deficiencies of prior devices, the art of coronary artery cannulation would benefit from an improved right transradial catheter taking into account anatomical relationships not yet exploited. For example, improvements that may be desirable are providing further support against cephalad back-up force in maintaining proper catheter engagement with the right coronary artery and improved catheter directionality. Regarding cephalad back-up force, a coronary procedure may require fluid to be introduced into an artery through a catheter that is aligned generally coaxial with the artery. Introduction of such fluid into the artery causes a force applied to the catheter, which may be generally in the cephalad direction. Unchecked, such force may cause disengagement of the catheter from the ostium of the artery. To prevent disengagement of a catheter from the ostium, further support may be desired.

### SUMMARY OF THE INVENTION

The present invention provides improved support against cephalad back-up force in maintaining proper catheter engagement with the right coronary artery and improved catheter directionality. P A catheter according to the present invention includes a distal end, a proximal end, and a tubular body extending through a body length between the distal end and proximal end. In one embodiment, the tubular body includes a tip extending from the distal end to a primary curve, a cephalic segment extending between the primary curve and a secondary curve, a subclavian segment extending between the secondary curve and a tertiary curve, an arm segment extending from the tertiary curve into a diagnostic segment. The diagnostic segment extends to the proximal end. The tubular body includes a lumen, which is accessible through the distal end, the proximal end, or both. P A catheter according to the present invention may be manufactured from a material that can be resiliently deformed to enable passage of the catheter through the radial artery and into the aortic arch and to the desired position within the ascending aorta. The material may include additional embedded braiding for added torqueability or stiffness. The additional embedded braiding may be provided at any position along the tubular body. In one embodiment, the embedded braiding is provided throughout the secondary curve. Alternatively, or in addition, braiding could be provided in the tertiary curve. A portion of the catheter may include a material that is at least partially radiopaque.

### BRIEF DESCRIPTION OF THE DRAWINGS

FIG. 1 is a side elevation view of an embodiment of a right transradial right coronary catheter according to the present invention. P FIG. 2 is a partial cutaway view of the embodiment of FIG. 1. P FIG. 3 is a side elevation view of a first prior device inserted a first distance into a patient's aorta. P FIG. 4 is a side elevation view of a second prior device inserted the same first distance into a patient's aorta as the distance in FIG. 3. P FIG. 5 is a side elevation view of an embodiment of a right transradial right coronary catheter according to the present invention inserted a second distance into a patient's aorta, the second distance being less than the first distance in FIGS. 3 and 4. P FIG. 6 is a side elevation view of the embodiment of FIG. 5 inserted the same first distance into a patient's aorta as in FIGS. 3 and 4. P FIG. 7 is a side elevation view of the embodiment of FIG. 5 having been situated at a desired position.

### DESCRIPTION OF THE PREFERRED EMBODIMENT

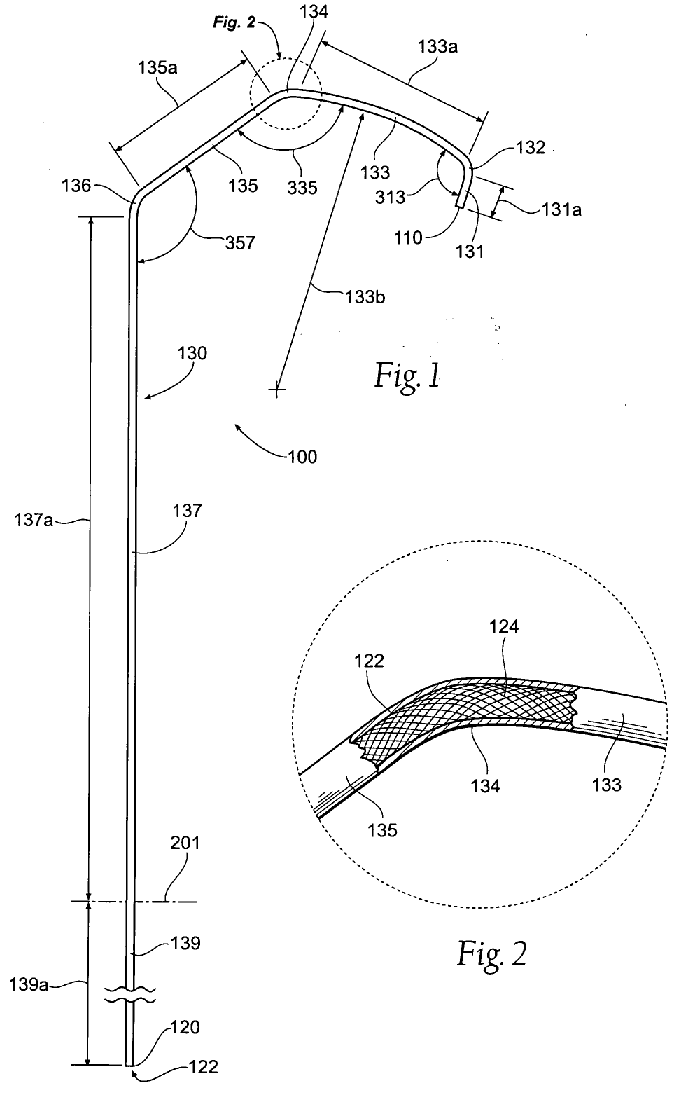

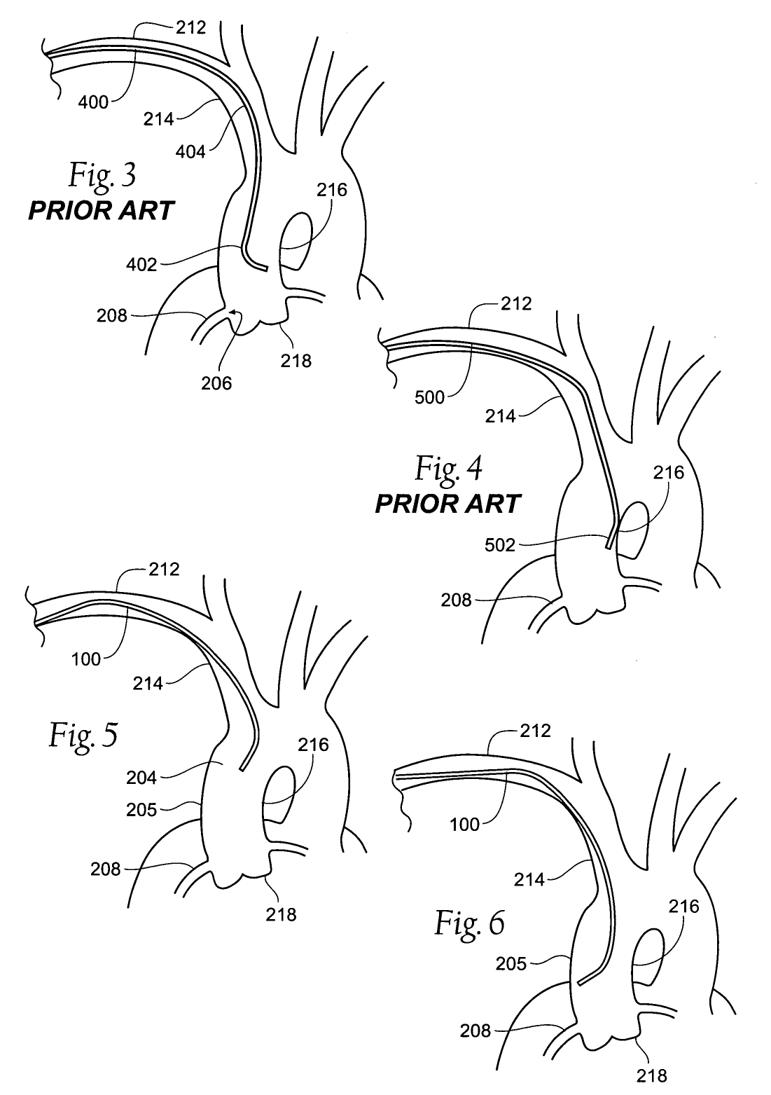

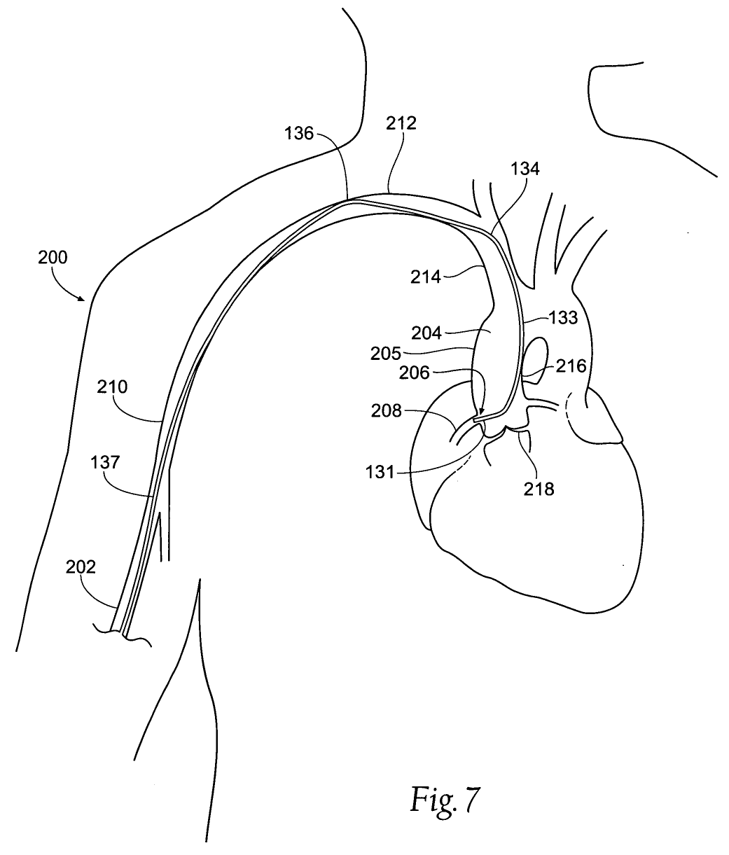

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims. P Turning now to the figures, FIG. 1 provides an embodiment 100 of a right transradial catheter according to the present invention. The embodiment 100 includes a distal end 110 a proximal end 120, and a tubular body 130 extending therebetween. The body 130 has a lumen 122 formed therethrough, which is accessible from the proximal end 110, the distal end 120, or both. The body 130 preferably has five body segments extending from the distal end 110 to the proximal end 120. The segments preferably include a tip 131, a cephalic segment 133, a subclavian segment 135, an arm segment 137, and a diagnostic segment 139. P The first four segments 131, 133, 135, 137 are preferably coupled to adjacent segments via three curved portions of the body 130. Although dimensions are provided herein, the dimensions are exemplary, and embodiment sizes of a catheter according to the present invention may be chosen with a target patient in mind. First, the tip 131, which has a length 131a of preferably about ten to about fifteen millimeters, is coupled to the cephalic segment 133 by way of a primary curved portion 132. The primary curved portion 132 provides an angle 313 between the tip 131 and the cephalic segment 133 of preferably about 90 to about 100 degrees. The cephalic segment 133, which has a length 133a of preferably about 100 to about 130 millimeters, is coupled to the subclavian segment 135 by way of a secondary curved portion 134. The secondary curved portion 134 provides an angle 335 between the cephalic segment 133 and the subclavian segment 135 of preferably about 130 degrees. Further, the cephalic segment 133 may be formed with a slight curvature between the first curved portion 132 and the second curved portion 134. A preferred radius 133b of such curvature may be about 160 to about 200 millimeters. The subclavian segment 135, which has a length 135a of preferably about 95 to about 105 millimeters, is coupled to the arm segment 137 by way of a tertiary curved portion 136. The tertiary curved portion 136 provides an angle 357 between the subclavian segment 135 and the arm segment 137 of preferably about 120 to about 130 degrees. The arm segment 137 has a length 137a of preferably about 315 to about 345 millimeters. The diagnostic segment 139, which may simply be an extension of the arm segment 137, has a length 139 that is preferably about 450 millimeters. The diagnostic segment 139 may be the portion of the catheter 100 that protrudes externally from the skin 201 of a patient. P The body 130 of an embodiment of a catheter according to the present invention is preferably manufactured from a material that can be resiliently deformed to enable passage of the catheter through the radial artery and into the aortic arch and to the desired position within the ascending aorta, having an at-rest formation as described. Such material is known in the art and can be found, for example, in U.S. Pat. No. 5,403,292, to Ju. Additionally, along certain portions of the body 132, where additional stiffness is desired, such stiffness may be obtained, for example, by additional braiding 124 added to the material forming the catheter or otherwise, for example, by quality of the catheter material at the appropriate regions. Such braiding 124 may be seen in FIG. 2. Additionally, the catheter 100 may be at least partially radiopaque, so as to allow examination of insertion progress and placement by, for example, X-ray. P In use, an embodiment 100 of a right transradial right coronary artery catheter according to the present invention may be inserted into a body 200 in a conventional transradial catheterization procedure in which the catheter 100 is inserted into the radial artery 202 of the wrist (not shown) and advanced into the ascending aorta 204. It is understood, however, that catheters according to the present invention may be used for other procedures, including brachial catheterizations. A detailed explanation of a transradial catheterization procedure may be found in U.S. Pat. No. 6,355,026 to Mick, mentioned and incorporated by reference above. Generally, to insert the catheter 100 into the aorta 204 of a patient 200, an access point is formed in the wrist by way of a puncture into the radial artery 202. A guidewire (not shown), as known in the art, is used to aid insertion and is removed once the catheter 100 is positioned desirably. P A catheter according to the present invention simplifies the catheterization procedure, as can be seen by analysis of prior devices during insertion. For example, FIG. 3 depicts a prior Judkins catheter 400 having been inserted a first distance into a patient's aorta 204, naturally deflecting without any torque applied to the catheter 400 by a physician outside the patient's body. Due to the alternating primary 402 and secondary 404 curves, the Judkins catheter 400 remains directed towards the left coronary cusp 218 rather than being directed towards the target, which in this case is the ostium 206 of the right coronary artery 208. Due to this alternating curvature, proper positioning of the Judkins catheter 400 in a right coronary artery requires significant torque to be applied to the catheter 400 external to the patient. P One improvement has been made in light of the Judkins catheter for use in a right transradial approach to the right coronary artery. That is, U.S. Pat. No. 6,355,026, to Mick, describes a right transradial coronary catheter that removes the alternating curvature included on the Judkins catheter. Despite removal of the alternating Judkins curvature, the tip 502 of the catheter 500 proceeds towards the left wall 216 of the ascending aorta 204, as shown in FIG. 4, herein, when the Mick catheter 500 is inserted the same distance as the Judkins catheter 400 in FIG. 3. Indeed, the contact of the Mick catheter 500 with the left wall 216 of ascending aorta 204 creates the biasing effect necessary to maintain the catheter 500 in coaxial engagement with the right coronary artery 208. P Improved directionality is offered by a catheter according to the present invention. FIG. 5 shows a catheter 100 according to the present invention having been introduced into the aorta 204 of a patient a distance less than the distance in FIGS. 3 and 4. FIG. 6 shows the same catheter 100 as FIG. 5 having been inserted the same distance as the prior catheters 400, 500 of FIGS. 3 and 4. As can be seen, the catheter 100 offers more targeted directionality towards the ostium 206 of the right coronary artery 208. That is, the tip 131 of the catheter 100 is maintained in relatively close proximity to the right wall 205 of the ascending aorta as it progresses into the aorta 204 from the bracheocephalic trunk 214, instead of allowing drift of the tip 131 towards the left coronary cusp 218. This targeted directionality is enabled at least partially by the secondary curve 134 and the tertiary curve 136, and associated structure, such as reinforcement braiding 124 that may be provided therethrough. P A proper placement of a catheter 100 according to the present invention is shown in FIG. 7. The catheter 100 is shown as having its tip 131 positioned in the ostium 206 of a right coronary artery 208, after having been introduced into the radial artery 202, through the brachial artery 210, the right subclavian artery 212, the bracheocephalic trunk 214, and into the aorta 204. With the tip 131 placed in the ostium 206 of the right coronary artery 208, the cephalic segment 133 rests gently, if at all, against the left wall 216 of the ascending aorta 204. Also in this engaged position, the secondary curved portion 134 rests generally near the junction of the bracheocephalic trunk 214 and right subclavian artery 212, and the tertiary curved portion 136 rests generally in the proximal portion of the right subclavian artery 212. The secondary 134 and tertiary 136 curved portions thus preferably provide the primary catheter support and directionality to the cephalic segment 133 and the tip 131. In addition to improved directionality, the tertiary curve 136 offers additional resistance against cephalic, or upward, back-up force by providing caudal, or downward, torque to the brachiocephalic segment 135 and other distal portions of the catheter body 130. P The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A catheter comprising:
a distal end;
a proximal end;
a tubular body extending through a body length between said distal end and said proximal end;
said tubular body comprising:
a tip extending from the distal end to a primary curve, said primary curve being bent in a first direction;
a cephalic segment extending from said primary curve to a secondary curve, said secondary curve being bent in said first direction, wherein said cephalic segment has a length of about 105 to about 125 millimeters and said cephalic segment is curved along at least a majority of said length, wherein the curve of said cephalic segment has a radius of about 160 millimeters to about 200 millimeters;
a subclavian segment extending from said secondary curve to a tertiary curve, said tertiary curve being bent in said first direction;
an arm segment extending from said tertiary curve into a diagnostic segment;
and said diagnostic segment extending to said proximal end.

2. A catheter according to claim 1, said tubular body including a lumen.

3. A catheter according to claim 2, said lumen being accessible through said proximal end.

4. A catheter according to claim 3, said lumen being accessible through said distal end.

5. A catheter according to claim 2, said lumen being accessible through said distal end.

6. A catheter according to claim 1, said tip having a length of about 10 millimeters to about 15 millimeters.

7. (canceled)

8. A catheter according to claim 1, said cephalic segment being curved along substantially all of said length of said cephalic segment.

9. A catheter according to claim 1, said primary curve providing an angle of about 90 to about 100 degrees between said tip and said cephalic segment.

10. A catheter according to claim 1, said secondary curve providing an angle of about 125 to about 135 degrees between said cephalic segment and said subclavian segment.

11. A catheter according to claim 10, said secondary curve providing an angle of 130 degrees between said cephalic segment and said subclavian segment.

12. A catheter according to claim 1, said tertiary curve providing an angle of about 120 to about 135 degrees between said subclavian segment and said arm segment.

13. A catheter according to claim 1, said catheter at least partially comprising a resilient polyamide material.

14. A catheter according to claim 13, said catheter comprising reinforcement braiding in at least one predetermined location.

15. A catheter according to claim 14, said at least one predetermined location comprising said secondary curve.

16. A catheter according to claim 14, said at least one predetermined location comprising said tertiary curve.

17. A catheter according to claim 1, wherein a portion of said catheter comprises a material which is at least partially radiopaque.

18. A method of cannulating a right coronary artery of a human through a right radial artery of said human, the method comprising the steps of:
providing a catheter comprising:
a distal end;
a proximal end;
a tubular body extending through a body length between said distal end and said proximal end;
said tubular body comprising:
a tip extending from the distal end to a primary curve, said primary curve being bent in a first direction;
a cephalic segment extending from said primary curve to a secondary curve, said secondary curve being bent in said first direction, wherein said cephalic segment has a length of about 105 to about 125 millimeters and said cephalic segment is curved along at least a majority of said length, wherein the curve of said cephalic segment has a radius of about 160 millimeters to about 200 millimeters;
a subclavian segment extending from said secondary curve to a tertiary curve, said tertiary curve being bent in said first direction;
an arm segment extending from said tertiary curve into a diagnostic segment, said diagnostic segment extending to said proximal end;
forming an access point in said right radial artery of said human;
inserting said tip of said catheter through said access point and into said right radial artery;
guiding said tip through said right radial artery and into the right brachial artery of said human;
guiding said tip through said right brachial artery and into the right subclavian artery of said human;
guiding said tip through said right subclavian artery of said human and into the bracheocephalic trunk of said human;
guiding said tip through said bracheocephalic trunk and into the ascending aorta of said human;
guiding said tip through said ascending aorta and into the ostium of said right coronary artery of said human; and
resting said tertiary curve in said right subclavian artery closer to said right brachial artery than to said bracheocephalic trunk.
